(19) United States
(12) Patent Application Publication
(10) Pub. No.: US 2013/0129676 A1
(43) Pub. Date: May 23, 2013

(54) COMBINATION OF ANTI-ENVELOPE ANTIBODIES AND ANTI-RECEPTOR ANTIBODIES FOR THE TREATMENT AND PREVENTION OF HCV INFECTION

(71) Applicant: Thomas Baumert, Strasbourg (FR)

(72) Inventor: Thomas Baumert, Strasbourg (FR)

(73) Assignees: UNIVERSITE DE STRASBOURG, Strasbourg (FR); INSERM (Institut National de la Sante et de la Recherche Medicale), Paris (FR)

(21) Appl. No.: 13/699,060

(22) PCT Filed: May 25, 2011
(86) PCT No.: PCT/EP2011/058538

§ 371 Date: Feb. 4, 2013

(30) Foreign Application Priority Data
May 25, 2010 (EP) 10305546.3

(51) Int. Cl. A61K 39/42; A61K 45/06; A61K 31/7088; A61K 38/21; A61K 31/7056

(52) U.S. Cl. CPC A61K 39/42; A61K 38/21; A61K 31/7056; A61K 31/7088; A61K 45/06
USPC 424/85.4; 424/161.1; 424/149.1; 424/133.1; 424/178.1

(57) ABSTRACT

The present invention provides combinations of antibodies for use in the treatment or the prevention of HCV infection. In particular, combinations are provided that comprise at least one anti-HCV envelope antibody and at least one anti-HCV receptor antibody, wherein the anti-HCV-envelope antibody and anti-HCV-receptor antibody act in a highly synergistic manner to inhibit HCV entry into susceptible cells. Also provided are pharmaceutical compositions and kits comprising such combinations and methods of using these compositions and kits for treating or preventing HCV infection.

### RELATED APPLICATION

The present application claims priority to European Patent Application No. EP 10 305 546 filed on May 25, 2010, which is incorporated herein by reference in its entirety.

### BACKGROUND OF THE INVENTION

Hepatitis C virus (HCV) is a major global health problem, with an estimated 150-200 million people infected worldwide, including at least 5 millions within Europe (Pawlotsky, Trends Microbiol., 2004, 12: 96-102). According to the World Health Organization, 3 to 4 million new infections occur each year. The infection is often asymptomatic; however, the majority of HCV-infected individuals develop chronic infection (Hoofnagle, Hepatology, 2002, 36: 521-S29; Lauer et al., N. Engl. J. Med., 2001, 345: 41-52; Seeff, Semin. Gastrointest., 1995, 6: 20-27). Chronic infection frequently results in serious liver disease, including fibrosis and steatosis (Chisari, Nature, 2005, 435: 930-932). About 20% of patients with chronic HCV infection develop liver cirrhosis, which progresses to hepatocellular carcinoma in 5% of the cases (Hoofnagle, Hepatology, 2002, 36: S21-S29).

Chronic HCV infection is the leading indication for liver transplantations (Seeff et al., Hepatology, 2002, 36: 1-2). Unfortunately, liver transplantation is not a cure for hepatitis C; viral recurrence is an invariable problem and the leading cause of graft loss (Brown, Nature, 2005, 436: 973-978). No vaccine protecting against HCV is yet available. Current therapies include administration of ribavirin and/or interferon-alpha (IFN-α), two non-specific anti-viral agents. Using a combination treatment of pegylated IFN-α and ribavirin, persistent clearance is achieved in about 50% of patients with chronic hepatitis C infected with genotype 1. However, a large number of patients have contraindications to one of the components of the combination, cannot tolerate the treatment, do not respond to IFN therapy at all or experience a relapse when administration is stopped. In addition to limited efficacy and substantial side effects such as neutropenia, haemolytic anemia and severe depression, current antiviral therapies are also characterized by high cost.

HCV entry into target cells is a promising target for antiviral preventive and therapeutic strategies since it is essential for initiation, spread, and maintenance of infection (Timpe et al., Gut, 2008, 57: 1728-1737; Zeisel et al., Hepatology, 2008, 48: 299-307). Indeed, HCV initiates infection by attaching to molecules or receptors on the surface of hepatocytes. Current evidence suggests that HCV entry is a multistep process involving several host factors including heparan sulfate (Barth et al., J. Biol. Chem., 2003, 278: 41003-41012), the tetraspanin CD81 (Pileri et al., Science, 1998, 282: 938-941), the scavenger receptor class B type I (SB-RI) (Zeisel et al., Hepatology, 2007, 46: 1722-1731; Bartosch et al., J. Exp. Med., 2003, 197: 633-642; Grove et al., J. Virol., 2007, 81: 3162-3169; Kapadia et al., J. Virol., 2007, 81: 374-383; Scarselli et al., EMBO J., 2002, 21: 5017-5025), Occludin (Ploss et al., Nature, 2009, 457: 882-886) and Claudin-1 (CLDN1), an integral membrane protein and a component of tight junction strands (Evans et al., Nature, 2007, 446: 801-805).

Identification of these receptors or co-receptors for HCV has opened up new avenues for the development of therapeutic and prophylactic agents as drug candidates for the prevention and/or treatment of HCV infection. Thus, cross-neutralizing antibodies inhibiting HCV entry have been shown to be associated with control of HCV infection and prevention of HCV re-infection in cohorts with self-limited acute infection (Osburn et al., Gastroenterology, 2009, 138: 315-324; Pestka et al., Proc. Natl. Acad. Sci. USA, 2007, 104: 6025-630). For example, monoclonal antibodies have been generated against native human SR-BI that inhibit HCV E2 binding to SR-BI and efficiently block HCVcc infection of hepatoma cells in a dose-dependent manner (Catanese et al., J. Virol., 2007, 81: 8063-8071; WO 2006/005465). European patent application No. EP 1 256 348 discloses substances, including antibodies, with antiviral effects that inhibit binding of HCV E2 and CD81. International patent application WO 2007/130646 describes in vitro and cell-based assays for identifying agents that interfere with HCV interactions with Claudin-1 thereby preventing HCV infection. Monoclonal antibodies have been generated that efficiently inhibit HCV infection by targeting host entry factor Claudin-1 (EP 08 305 597 and WO 2010/034812).

Other studies have shown that cross-neutralizing anti-E2 antibodies or purified heterologous anti-HCV IgG obtained from chronically infected patients are capable of neutralizing genetically diverse HCV isolates and could protect against HCV quasispecies challenge (Meunier et al., Proc. Natl. Acad. Sci. USA, 2005, 102: 4560-4565; Law et al., Nat. Med., 2008, 14: 25-27; Broering et al., J; Virol., 2009, 83: 12473-12482). The administration of exogenous cross-neutralizing anti-envelope antibodies has been proposed as another immunotherapeutic approach for the prevention of HCV re-infection. The ability of polyclonal immunoglobulin preparation enriched in anti-HCV is currently evaluated in a phase II study to assess the therapeutic benefit to prevent HCV infection after liver transplantation (Clinical Trials Identifier: NCT00473824).

Since the development of novel therapeutic approaches against HCV remains a high-priority goal, these studies are encouraging as they demonstrate that antibodies against receptor or co-receptors that affect HCV entry into susceptible cells may constitute an effective and safe alternative to current HCV therapies.

### SUMMARY OF THE INVENTION

The present invention relates to targeted systems and improved strategies for the prevention and/or treatment of HCV infection and HCV-related diseases. More specifically, the present Applicants have demonstrated that a cross-neutralizing anti-envelope (anti-E2) antibody or a purified heterologous anti-HCV IgG and an anti-CLND1 antibody act in a highly synergistic manner on the inhibition of entry of highly infectious HCV escape variants (see Example 1), suggesting that the combination of cross-neutralizing anti-HCV-envelope antibodies and anti-HCV-receptor antibodies is an effective antiviral approach to prevent primary HCV infection, such as after liver transplantation and might also restrain virus spread in chronically infected patients.

Consequently, in one aspect, the present invention provides a combination of at least one anti-HCV-envelope antibody and at least one anti-HCV-receptor antibody for use in the treatment or prevention of HCV infection.

In certain preferred embodiments, the anti-HCV-envelope antibody is an anti-HCV-envelope glycoprotein antibody such as an anti-E1 antibody or an anti-E2 antibody. The anti-HCV-envelope antibody may also be an anti-HCV IgG isolated and purified from a human individual previously or chronically infected with HCV. The anti-HCV-envelope antibody may be a polyclonal antibody or a monoclonal antibody. Preferably, the anti-HCV-envelope antibody is a monoclonal antibody.

The anti-HCV-receptor antibody of a combination according to the invention may be an antibody against any HCV receptor known in the art or an antibody directed against any cell surface protein involved in the HCV infection process. The anti-HCV-receptor antibody may be a polyclonal antibody or a monoclonal antibody. Preferably, the anti-HCV-receptor antibody is a monoclonal antibody. In certain embodiments, the anti-HCV-receptor antibody is an antibody against a receptor selected from the group consisting of heparan sulfate, the LDL receptor, the tetraspanin CD81, the scavenger receptor class B type I (SB-RI), Occludin and Claudin-1 (CLDN1). In certain preferred embodiments, the anti-HCV-receptor antibody is an anti-CLDN1 antibody, in particular a monoclonal anti-CLDN1 antibody such as those developed by the present Applicants and described in EP 08 305 597 and WO 2010/034812.

The antibodies of a combination according to the present invention may be full (complete) antibodies, or biologically active fragment of such antibodies (i.e., any fragment or portion of such an antibody that retains the ability of the antibody to interfere with HCV-host cells interactions, and/or to specifically bind to a HCV receptor or to a HCV envelope protein, and/or to inhibit or block HCV entry into HCV-susceptible cells, and/or to reduce or prevent HCV infection of susceptible cells). Antibodies or fragments thereof that are suitable for use in a combination according to the present invention also include chimeric antibodies, humanized antibodies, de-immunized antibodies and antibody-derived molecules comprising at least one complementary determining region (CDR) from either a heavy chain or light chain variable region of an anti-HCV-receptor antibody or an anti-HCV-envelope antibody, including molecules such as Fab fragments, F(ab′)₂ fragments, Fd fragments, Sc antibodies (single chain antibodies), diabodies, individual antibody light single chains, individual antibody heavy chains, chimeric fusions between antibody chains and other molecules, and antibody conjugates, such as antibodies conjugated to a therapeutic agent, so long as these antibody-related molecules retain at least one biologically relevant property of the antibody from which it is “derived”. The biologically relevant property may be the ability to interfere with HCV-host cells interactions, to specifically bind to an HCV envelope protein or to an HCV receptor, to inhibit or block HCV entry into HCV-susceptible cells, and/or to reduce or prevent HCV infection of susceptible cells.

In a combination according to the present invention, the anti-HCV-envelope antibody and anti-HCV-receptor antibody act in a highly synergistic manner on the inhibition of HCV infection. In certain embodiments, the anti-HCV-envelope antibody decreases the IC₅₀ for the inhibition of HCV infection by the anti-HCV-receptor antibody by a factor of up to at least 10 fold or up to at least 25 fold, preferably up to at least 50 fold, more preferably up to at least 75 fold, and even more preferably up to 100 fold. In other embodiments, the anti-HCV-receptor antibody decreases the IC₅₀ for the inhibition of HCV infection by the anti-HCV-envelope antibody by a factor of up to at least 10 fold or up to at least 25 fold, preferably up to at least 50 fold, more preferably up to at least 75 fold, and even more preferably up to 100 fold. The combination index (CI) of the at least one anti-HCV-envelope antibody and at least one anti-HCV-receptor antibody is lower than 1, preferably lower than 0.75, more preferably lower than 0.50, and even more preferably lower than 0.30.

The combinations of the present invention can find application in a variety of prophylactic and therapeutic treatments. Thus, the combinations are provided for use in the prevention of HCV infection of a cell (e.g., a susceptible cell or a population of susceptible cells); for preventing or treating HCV infection or a HCV-related disease in a subject; for controlling chronic HCV infection; and for preventing HCV recurrence in a liver transplantation patient. HCV infection may be due to HCV of a genotype selected from the group consisting of genotype 1, genotype 2, genotype 3, genotype 4, genotype 5 and genotype 6, or more specifically of a subtype selected from the group consisting of subtype 1a, subtype 1b, subtype 2a, subtype 2b, subtype 2c, subtype 3a, subtype 4a-f, subtype 5a, and subtype 6a.

In a related aspect, the present invention provides a method of reducing the likelihood of a susceptible cell of becoming infected with HCV as a result of contact with HCV, which comprises contacting the susceptible cell with an effective amount of an inventive combination. Also provided is a method of reducing the likelihood of a subject's susceptible cells of becoming infected with HCV as a result of contact with HCV, which comprises administering to the subject an effective amount of an inventive combination. The present invention also provides a method of treating or preventing HCV infection or a HCV-associated disease (e.g., a liver disease or pathology) in a subject in need thereof, which comprises administering to the subject an effective amount of an inventive combination. The invention also provides a method for controlling chronic HCV infection in a subject in need thereof, which comprises administering to the subject an effective amount of an inventive combination.

Also provided is a method of preventing HCV recurrence in a liver transplantation patient, which comprises administering to the patient an effective amount of an inventive combination. Administration of an inventive combination to a subject may be by any suitable route, including, for example, parenteral, aerosol, oral and topical routes. The inventive combination may be administered alone or in combination with a therapeutic agent, such as an anti-viral agent.

The inventive combinations may be administered per se or as pharmaceutical compositions. Accordingly, in another aspect, the present invention provides for the use of an inventive combination for the manufacture of medicaments, pharmaceutical compositions, or pharmaceutical kits for the treatment and/or prevention of HCV infection and HCV-associated diseases.

In a related aspect, the present invention provides a pharmaceutical composition comprising an effective amount of an inventive combination (i.e., at least one anti-HCV-envelope antibody and at least one anti-HCV-receptor antibody as described herein) and at least one pharmaceutically acceptable carrier or excipient. In certain embodiments, the pharmaceutical composition is adapted for administration in combination with an additional therapeutic agent, such as an antiviral agent. In other embodiments, the pharmaceutical composition further comprises an additional therapeutic agent, such as an antiviral agent. Antiviral agents suitable for use in methods and pharmaceutical compositions of the present invention include, but are not limited to, interferons (e.g., interferon-alpha, pegylated interferon-alpha), ribavirin, anti-HCV (monoclonal or polyclonal) antibodies, RNA polymerase inhibitors, protease inhibitors, IRES inhibitors, helicase inhibitors, antisense compounds, ribozymes, entry inhibitors, and any combination thereof.

These and other objects, advantages and features of the present invention will become apparent to those of ordinary skill in the art having read the following detailed description of the preferred embodiments.

### BRIEF DESCRIPTION OF THE DRAWING

FIG. 1 is a set of three graphs illustrating the synergistic effect of antiviral and anti-CLDN1 antibodies on the inhibition of HCVpp infection. HCVpp of strains P02VJ and P04VJ were pre-incubated with anti-E1 mAb IGH526 (A), anti-E2 mAb IGH461 (B) or purified heterologous anti-HCV IgG (1 or 10 μg/ml) obtained from an unrelated chronically infected subject (C) or isotype control IgG for 1 hour at 37° C. and added to Huh7 cells pre-incubated with serial dilutions of anti-CLDN1 OM-7D3-B3 or rat isotype control mAbs. HVCpp infection was analyzed as described in Example 1 by quantification of luciferase reporter gene expression.

FIG. 2 is a set of three graphs illustrating the synergistic effect of antiviral and anti-SR-BI antibodies on the inhibition of HCVpp infection. HCVpp of strains P02VJ and P04VJ were pre-incubated with anti-E1 mAb IGH526 (A), anti-E2 mAb IGH461 (B) or purified heterologous anti-HCV IgG (1 or 10 μg/ml) obtained from an unrelated chronically infected subject (C) or isotype control IgG for 1 hour at 37° C. and added to Huh7 cells pre-incubated with serial dilutions of anti-SR-BI NK-8H5-E3 or mouse isotype control mAbs. HVCpp infection was analyzed as described in Example 1 by quantification of luciferase reporter gene expression.

FIG. 3 is a set of three graphs illustrating the synergistic effect of antiviral and anti-CD81 antibodies on the inhibition of HCVpp infection. HCVpp of strains P02VJ and P04VJ were pre-incubated with anti-E1 mAb IGH526 (A), anti-E2 mAb IGH461 (B) or purified heterologous anti-HCV IgG (1 or 10 μg/ml) obtained from an unrelated chronically infected subject (C) or isotype control IgG for 1 hour at 37° C. and added to Huh7 cells pre-incubated with serial dilutions of anti-CD81 QV-6A8-F2CA or rat isotype control mAbs. HVCpp infection was analyzed as described in Example 1 by quantification of luciferase reporter gene expression.

FIG. 4 is a set of three graphs illustrating the synergistic effect of antiviral and anti-HCV-receptor antibodies on cell-culture-derived HCV (HCVcc). HCVcc (Luc-Jc1, genotype 2a) obtained from an unrelated chronically infected subject or isotype control IgG for 1 hour at 37° C. and added to Huh7 cells pre-incubated with serial dilutions of anti-CLDN1 OM-7D3-B3 (A), anti-SR-BI NK-8H5-E3 (B), anti-CD81 QV-6A8-F2CA (C), rat or mouse isotype control mAbs. HVCcc infection was analyzed as described in Example 2 by quantification of luciferase reporter gene expression.

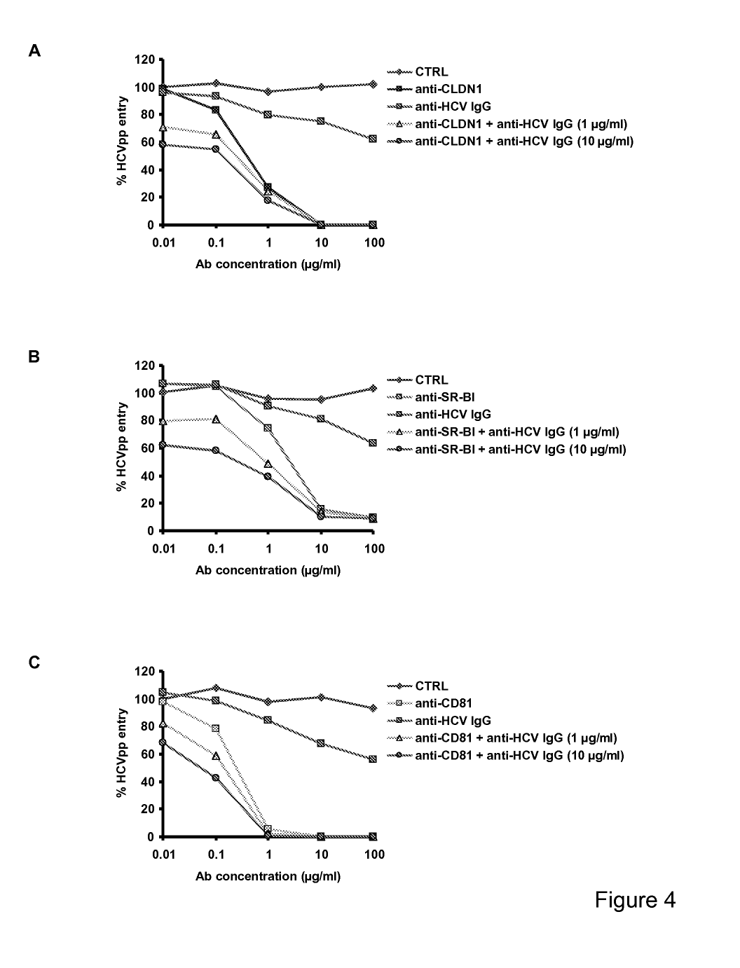

### DEFINITIONS

Throughout the specification, several terms are employed that are defined in the following paragraphs.

As used herein, the term “subject” refers to a human or another mammal (e.g., primate, dog, cat, goat, horse, pig, mouse, rat, rabbit, and the like), that can be the host of Hepatitis C virus (HCV), but may or may not be infected with the virus, and/or may or may not suffer from a HCV-related disease. Non-human subjects may be transgenic or otherwise modified animals. In many embodiments of the present invention, the subject is a human being. In such embodiments, the subject is often referred to as an “individual”. The term “individual” does not denote a particular age, and thus encompasses newborns, children, teenagers, and adults.

As used herein, the term “HCV” refers to any major HCV genotype, subtype, isolate and/or quasispecies. HCV genotypes include, but are not limited to, genotypes 1, 2, 3, 4, 5, and 6; HCV subtypes include, but are not limited to, subtypes 1a, 1b, 2a, 2b, 2c, 3a, 4a-f, 5a and 6a.

The terms “afflicted with HCV” or “infected with HCV” are used herein interchangeably. When used in reference to a subject, they refer to a subject that has at least one cell which is infected by HCV. The term “HCV infection” refers to the introduction of HCV genetic information into a target cell, such as by fusion of the target cell membrane with HCV or an HCV envelope glycoprotein-positive cell.

The terms “HCV-related disease” and “HCV-associated disease” are herein used interchangeably. They refer to any disease or disorder known or suspected to be associated with and/or caused, directly or indirectly, by HCV. HCV-related (or HCV-associated) diseases include, but are not limited to, a wide variety of liver diseases, such as subclinical carrier state of acute hepatitis, chronic hepatitis, cirrhosis, and hepatocellular carcinoma. The term includes symptoms and side effects of any HCV infection, including latent, persistent and sub-clinical infections, whether or not the infection is clinically apparent.

The term “treatment” is used herein to characterize a method or process that is aimed at (1) delaying or preventing the onset of a disease or condition (e.g., HCV infection or HCV-related disease); (2) slowing down or stopping the progression, aggravation, or deterioration of the symptoms of the disease or condition; (3) bringing about amelioration of the symptoms of the disease or condition; or (4) curing the disease or condition. A treatment may be administered prior to the onset of the disease or condition, for a prophylactic or preventive action. Alternatively or additionally, a treatment may be administered after initiation of the disease or condition, for a therapeutic action.

A “pharmaceutical composition” is defined herein as comprising an effective amount of at least one antibody (or a fragment thereof) of the invention, and at least one pharmaceutically acceptable carrier or excipient.

As used herein, the term “effective amount” refers to any amount of a compound, agent, antibody, or composition that is sufficient to fulfil its intended purpose(s), e.g., a desired biological or medicinal response in a cell, tissue, system or subject. For example, in certain embodiments of the present invention, the purpose(s) may be: to prevent HCV infection, to prevent the onset of a HCV-related disease, to slow down, alleviate or stop the progression, aggravation or deterioration of the symptoms of a HCV-related disease (e.g., chronic hepatitis C, cirrhosis, and the like); to bring about amelioration of the symptoms of the disease, or to cure the HCV-related disease.

The term “pharmaceutically acceptable carrier or excipient” refers to a carrier medium which does not interfere with the effectiveness of the biological activity of the active ingredient(s) and which is not excessively toxic to the host at the concentration at which it is administered. The term includes solvents, dispersion, media, coatings, antibacterial and antifungal agents, isotonic agents, and adsorption delaying agents, and the like. The use of such media and agents for pharmaceutically active substances is well known in the art (see for example “Remington's Pharmaceutical Sciences”, E. W. Martin, 18^{th} Ed., 1990, Mack Publishing Co.: Easton, Pa., which is incorporated herein by reference in its entirety).

The term “antibody”, as used herein, refers to any immunoglobulin (i.e., an intact immunoglobulin molecule, an active portion of an immunoglobulin molecule, etc.) that binds to a specific epitope. The term encompasses monoclonal antibodies and polyclonal antibodies. All derivatives and fragments thereof, which maintain specific binding ability, are also included in the term. The term also covers any protein having a binding domain, which is homologous or largely homologous to an immunoglobulin-binding domain. These proteins may be derived from natural sources, or partly or wholly synthetically produced.

The term “specific binding”, when used in reference to an antibody, refers to an antibody binding to a predetermined antigen. Typically, the antibody binds with an affinity of at least 1×10⁷ M⁻¹, and binds to the predetermined antigen with an affinity that is at least two-fold greater than the affinity for binding to a non-specific antigen (e.g., BSA, casein).

The term “human Claudin-1 or human CLDN1” refers to a protein having the sequence shown in NCBI Accession Number NP_{—}066924, or any naturally occurring variants commonly found in HCV permissive human populations. The term “extracellular domain” or “ectodomain” of Claudin-1 refers to the region of the Claudin-1 sequence that extends into the extracellular space.

The terms “susceptible cell” and “HCV-susceptible cell” are used interchangeably. They refer to any cell that may be infected with HCV. Susceptible cells include, are not limited to, liver or hepatic cells, primary cells, hepatoma cells, CaCo2 cells, dendritic cells, placental cells, endometrial cells, lymph node cells, lymphoid cells (B and T cells), peripheral blood mononuclear cells, and monocytes/macrophages.

The term “preventing, inhibiting or blocking HCV infection” when used in reference to an inventive antibody or antibody-related molecule, means reducing the amount of HCV genetic information introduced into a susceptible cell or susceptible cell population as compared to the amount that would be introduced in the absence of the antibody or antibody-related molecule.

The term “isolated”, as used herein in reference to a protein or polypeptide, means a protein or polypeptide, which by virtue of its origin or manipulation is separated from at least some of the components with which it is naturally associated or with which it is associated when initially obtained. By “isolated”, it is alternatively or additionally meant that the protein or polypeptide of interest is produced or synthesized by the hand of man.

The terms “protein”, “polypeptide”, and “peptide” are used herein interchangeably, and refer to amino acid sequences of a variety of lengths, either in their neutral (uncharged) forms or as salts, and either unmodified or modified by glycosylation, side-chain oxidation, or phosphorylation. In certain embodiments, the amino acid sequence is a full-length native protein. In other embodiments, the amino acid sequence is a smaller fragment of the full-length protein. In still other embodiments, the amino acid sequence is modified by additional substituents attached to the amino acid side chains, such as glycosyl units, lipids, or inorganic ions such as phosphates, as well as modifications relating to chemical conversions of the chains such as oxidation of sulfydryl groups. Thus, the term “protein” (or its equivalent terms) is intended to include the amino acid sequence of the full-length native protein, or a fragment thereof, subject to those modifications that do not significantly change its specific properties. In particular, the term “protein” encompasses protein isoforms, i.e., variants that are encoded by the same gene, but that differ in their pI or MW, or both. Such iso forms can differ in their amino acid sequence (e.g., as a result of allelic variation, alternative splicing or limited proteolysis), or in the alternative, may arise from differential post-translational modification (e.g., glycosylation, acylation, phosphorylation).

The term “analog”, as used herein in reference to a protein, refers to a polypeptide that possesses a similar or identical function as the protein but need not necessarily comprise an amino acid sequence that is similar or identical to the amino acid sequence of the protein or a structure that is similar or identical to that of the protein. Preferably, in the context of the present invention, a protein analog has an amino acid sequence that is at least 30%, more preferably, at least 35%, 40%, 45%, 50%, 55%, 60%, 65%, 70%, 75%, 80%, 85%, 90%, 95% or 99% identical to the amino acid sequence of the protein.

The term “fragment” or the term “portion”, as used herein in reference to a protein, refers to a polypeptide comprising an amino acid sequence of at least 5 consecutive amino acid residues (preferably, at least about: 10, 15, 20, 25, 30, 35, 40, 50, 60, 70, 80, 90, 100, 125, 150, 175, 200, 250 or more amino acid residues) of the amino acid sequence of a protein. The fragment of a protein may or may not possess a functional activity of the protein.

The term “biologically active”, as used herein to characterize a protein variant, analog or fragment, refers to a molecule that shares sufficient amino acid sequence identity or homology with the protein to exhibit similar or identical properties to the protein. For, example, in many embodiments of the present invention, a biologically active fragment of an inventive antibody is a fragment that retains the ability of the antibody to bind to the extracellular domain of Claudin-1.

The term “homologous” (or “homology”), as used herein, is synonymous with the term “identity” and refers to the sequence similarity between two polypeptide molecules or between two nucleic acid molecules. When a position in both compared sequences is occupied by the same base or same amino acid residue, the respective molecules are then homologous at that position. The percentage of homology between two sequences corresponds to the number of matching or homologous positions shared by the two sequences divided by the number of positions compared and multiplied by 100. Generally, a comparison is made when two sequences are aligned to give maximum homology. Homologous amino acid sequences share identical or similar amino acid sequences. Similar residues are conservative substitutions for, or “allowed point mutations” of, corresponding amino acid residues in a reference sequence. “Conservative substitutions” of a residue in a reference sequence are substitutions that are physically or functionally similar to the corresponding reference residue, e.g. that have a similar size, shape, electric charge, chemical properties, including the ability to form covalent or hydrogen bonds, or the like. Particularly preferred conservative substitutions are those fulfilling the criteria defined for an “accepted point mutation” as described by Dayhoff et al. (“Atlas of Protein Sequence and Structure”, 1978, Nat. Biomed. Res. Foundation, Washington, D.C., Suppl. 3, 22: 354-352).

The terms “approximately” and “about”, as used herein in reference to a number, generally include numbers that fall within a range of 10% in either direction of the number (greater than or less than the number) unless otherwise stated or otherwise evident from the context (except where such number would exceed 100% of a possible value).

### DETAILED DESCRIPTION OF CERTAIN PREFERRED EMBODIMENTS

As mentioned above, the present invention provides combinations of antibodies for the treatment and prevention of HCV infection.

### I—Combinations

A combination according to the invention comprises at least one anti-HCV-envelope antibody and at least anti-HCV-receptor antibody, and is intended for use in the treatment or the prevention of HCV infection.

### A. Anti-HCV-Receptor Antibodies

The terms “anti-HCV receptor antibody”, and “anti-host factor antibody” are used herein interchangeably. They refer to any antibody raised against a receptor of HCV (or a region of a HCV receptor), in particular a receptor (or a region of a receptor) that is known to be involved in HCV entry into susceptible cells. They also refer to any antibody directed against a cell surface protein involved in the HCV infection, in particular in HCV entry into susceptible cells. Examples of such HCV receptors or cell surface proteins include heparan sulfate, the LDL receptor (Agnello et al., Proc. Natl. Acad. Sci. USA, 1999, 96: 12766-12771; Molina et al., J. Hepatol., 2007, 46: 411-419), the tetraspanin CD81, the scavenger receptor class B type I (SB-RI), Occludin and Claudin-1 (CLDN1).

Thus, anti-HCV-receptor antibodies that are suitable for use in the practice of the present invention include antibodies against a HCV receptor selected from the group consisting of heparan sulfate, the LDL receptor, CD81, SB-RI, Occludin and CLDN1 (or specific regions thereof). In certain preferred embodiments, the anti-HCV-receptor antibodies are antibodies against CD81, SB-RI or CLDN1 (or specific regions thereof).

Examples of anti-heparan sulfate antibodies that can be used in the practice of the present invention include, but are not limited to, the antibodies described or used in Kurup et al., J. Biol. Chem., 2007, 282: 21032-21042; Briani et al., J. Neurol. Sci., 2005, 229-230; U.S. Pat. Appln. No. 2009/0136964.

Examples of anti-LDL receptor antibodies that can be used in the practice of the present invention include, but are not limited to, the antibodies described or used in Agnello et al., Proc. Natl. Acad. Sci. USA, 1999, 96: 12766-12771; WO 01/68710; WO 2002/048388; U.S. Pat. Appln. No. US 2008/0213287, and antibodies commercially available, for example, from Amersham International (e.g., Clone C7).

Examples of anti-occludin antibodies that can be used in the practice of the present invention include, but are not limited to, the antibodies described or used in Tokunaga et al., J. Histochem. Cytochem., 2007, 55: 735-744.

Examples of anti-CD81 antibodies that can be used in the practice of the present invention include, but are not limited to, the antibodies described or used in Meuleman et al., Hepatology, 2008, 48: 1761-1769; Dijkstra et al., Exp. Neurol., 2006, 202: 57-66; Azorsa et al., J. Immunol. Methods, 1999, 229: 35-48.

Examples of anti-SB-RI antibodies that can be used in the practice of the present invention include, but are not limited to, the antibodies described or used in Haberstroh et al., Gastroenterology, 2008, 135: 1719-1728; Barth et al., J. Virol., 2008, 82: 3466-3479; Zeisel et al., Hepatology, 2007, 46: 1722-1731; Catanese et al., J. Virol., 2007, 81: 8063-8071; WO 2006/005465.

Examples of anti-CLDN1 antibodies that can be used in the practice of the present include, in particular, the polyclonal and monoclonal anti-CLDN1 antibodies that are disclosed in EP 08 305 597 and WO 2010/034812. As described in these documents, eight monoclonal antibodies have been produced by genetic immunization and shown to efficiently inhibit HCV infection by targeting the extracellular domain of CLDN1. Using an infectious HCV model system and primary human hepatocytes, these monoclonal anti-CLDN1 antibodies have been demonstrated to efficiently inhibit HCV infection of all major genotypes as well as highly variable HCV quasispecies in individual patients. Furthermore, these antibodies efficiently blocked entry of highly infectious HCV escape variants that were resistant to neutralizing antibodies in six patients with HCV re-infection during liver transplantation. The monoclonal anti-CLDN1 antibodies are called OM-4A4-D4, OM-7C8-A8, OM-6D9-A6, OM-7D4-C1, OM-6E1-B5, OM-3E5-B6, OM-8A9-A3, and OM-7D3-B3. Other suitable anti-CLDN1 antibodies are monoclonal antibodies secreted by any one of the hybridoma cell lines deposited by the Applicants at the DSMZ (Deutsche Sammlung von Mikro-organismen and Zellkuturen GmbH, Inhoffenstraβe 7 B, 38124 Braunschweig, Germany) on Jul. 29, 2008 under Accession Numbers DSM ACC2931, DSM ACC2932, DSM ACC2933, DSM ACC2934, DSM ACC2935, DSM ACC2936, DSM ACC2937, and DSM ACC2938 (described in EP 08 305 597 and WO 2010/034812).

Other suitable anti-CLDN1 antibodies include those disclosed in European Pat. No. EP 1 167 389 and U.S. Pat. No. 6,627,439.

Methods for the production and isolation of monoclonal antibodies from hybridoma cultures are well known in the art. Hybridoma cells are grown using standard methods, in suitable culture media such as, for example, D-MEM and RPMI-1640 medium. An anti-Claudin-1 monoclonal antibody can be recovered and purified from hybridoma cell cultures by protein A purification, ammonium sulphate or ethanol precipitation, acid extraction, anion or cation exchange chromatography, phosphocellulose chromatography, hydrophobic interaction chromatography, affinity chromatography, such as Protein A column, hydroxylapatite chromatography, lectin chromatography, or any suitable combination of these methods. High performance liquid chromatography (HPLC) can also be employed for purification.

### B. Anti-HCV-Envelope Antibodies

The terms “anti-HCV-envelope antibody”, “anti-HCV envelope protein antibody”, and “anti-viral antibody” are used herein interchangeably. They refer to any antibody raised against an HCV envelope protein (or a specific region thereof), in particular a HCV envelope protein (or a specific region thereof) known to be involved in the initiation of infection of susceptible cells by HCV infection. In certain preferred embodiments, the HCV envelope protein is an-envelope glycoprotein such as E1 or E2. The terms “anti-HCV-envelope antibody” and “anti-HCV envelope protein antibody” also encompass anti-HCV IgGs isolated and purified from human subjects previously infected with HCV or from patients chronically infected with HCV.

Thus, in certain embodiments, anti-HCV-envelope antibodies that are suitable for use in the practice of the present invention are antibodies against E1, E2 or a specific region thereof.

Examples of anti-E1 antibodies that can be used in the practice of the present invention include, but are not limited to, the antibodies described or used in El-Awady et al., World J. Gastroenterol., 2006, 12: 2530-2535; Keck et al., J. Virol., 2004, 78: 7257-7263; Meunier et al., J. Virol., 2008, 82: 966-973; Haberstroh et al., Gastroenterology, 2008, 135: 1719-1728; EP 1 845 108.

Examples of anti-E2 antibodies that can be used in the practice of the present invention include, but are not limited to, the antibodies described or used in Broering et al., J. Virol., 2009, 83: 12473-12482; Perotti et al., J. Virol., 2008, 82: 1047-1052; Allander et al., J. Gen. Virol., 2000, 81: 2451-2459; Hadlock et al., J. Virol., 2000, 74: 10407-10416; Haberstroh et al., Gastroenterology, 2008, 135: 1719-1728; EP 1 845 108; U.S. Pat. No. 6,747,136; U.S. Pat. No. 6,538,114; U.S. Pat. No. 6,951,646; U.S. Pat. No. 7,507,408.

In other embodiments, the anti-HCV-envelope antibodies that are suitable for use in the practice of the present invention are anti-HCV IgGs purified from previously infected or chronically infected HCV patients. Examples of such antibodies include, but are not limited to the polyclonal human hepatitis C immune globulin (Civacir®), which is currently being evaluated in a phase II study for the prevention of HCV infection after liver transplantation.

### C. Suitable Antibodies

The antibodies of a combination according to the present invention may be polyclonal antibodies or monoclonal antibodies. In certain preferred embodiments, the antibodies of an inventive combination are monoclonal antibodies.

Antibodies of a combination of the present invention may be prepared by any suitable method known in the art. For example, an anti-HCV-receptor or anti-HCV-envelope monoclonal antibody may be prepared by recombinant DNA methods. These methods generally involve isolation of the genes encoding the desired antibody, transfer of the genes into a suitable vector, and bulk expression in a cell culture system. The genes or DNA encoding the desired monoclonal antibody may be readily isolated and sequenced using conventional procedures (e.g., by using oligonucleotide probes that are capable of binding specifically to genes encoding the heavy and light chains of murine antibodies). Hybridoma cell lines may serve as a preferred source of such DNA. Suitable host cells for recombinant production of antibodies include, but are not limited to, appropriate mammalian host cells, such as CHO, HeLa, or CV1. Suitable expression plasmids include, without limitation, pcDNA3.1 Zeo, pIND(SP1), pREP8 (all commercially available from Invitrogen, Carlsboad, Calif., USA), and the like. The antibody genes may be expressed via viral or retroviral vectors, including MLV-based vectors, vaccinia virus-based vectors, and the like. The antibodies of a combination according to the present invention may be expressed as single chain antibodies. Isolation and purification of recombinantly produced antibodies may be performed by standard methods. Alternatively, antibodies of a combination of the present invention may be obtained from commercial sources.

In certain embodiments, an anti-HCV-receptor antibody or an anti-HCV-envelope antibody is used in its native form. In other embodiments, it may be truncated (e.g., via enzymatic cleavage or other suitable method) to provide immunoglobulin fragments or portions, in particular, fragments or portions that are biological active. Biologically active fragments or portions of an anti-HCV-receptor antibody or an anti-HCV-envelope antibody include fragments or portions that retain the ability of the antibody to interfere with HCV-host cells interactions, and/or to specifically bind the receptor or envelope, and/or to inhibit or block HCV entry into susceptible cells, and/or to reduce or prevent HCV infection of susceptible cells.

A biologically active fragment or portion of an anti-HCV-receptor antibody or an anti-HCV-envelope antibody may be an Fab fragment or portion, an F(ab′)₂ fragment or portion, a variable domain, or one or more CDRs (complementary determining regions) of the antibody. Alternatively, a biologically active fragment or portion of an anti-HCV-receptor antibody or an anti-HCV-envelope antibody may be derived from the carboxyl portion or terminus of the antibody protein and may comprise an Fc fragment, an Fd fragment or an Fv fragment.

Antibody fragments of the present invention may be produced by any suitable method known in the art including, but not limited to, enzymatic cleavage (e.g., proteolytic digestion of intact antibodies) or by synthetic or recombinant techniques. F(ab′)₂, Fab, Fv and ScFv (single chain Fv) antibody fragments can, for example, be expressed in and secreted from mammalian host cells or from E. coli. Antibodies can also be produced in a variety of truncated forms using antibody genes in which one or more stop codons have been introduced upstream of the natural stop site. The various portions of antibodies can be joined together chemically by conventional techniques, or can be prepared as a contiguous protein using genetic engineering techniques.

Anti-HCV-receptor and anti-HCV-envelope antibodies of a combination according to the present invention (or fragments thereof) may be produced in a modified form, such as a fusion protein (i.e., an immunoglobulin molecule or portion linked to a polypeptide entity). Preferably, the fusion protein retains the biological property of the antibody. A polypeptide entity to be fused to an anti-HCV-receptor or anti-HCV-envelope antibody, or a fragment thereof, may be selected to confer any of a number of advantageous properties to the resulting fusion protein. For example, the polypeptide entity may be selected to provide increased expression of the recombinant fusion protein. Alternatively or additionally, the polypeptide entity may facilitate purification of the fusion protein, for example, by acting as a ligand in affinity purification. A proteolytic cleavage site may be added to the recombinant protein so that the desired sequence can ultimately be separated from the polypeptide entity after purification. The polypeptide entity may also be selected to confer an improved stability to the fusion protein, when stability is a goal. Examples of suitable polypeptide entities include, for example, polyhistidine tags, that allow for the easy purification of the resulting fusion protein on a nickel chelating column. Glutathione-S-transferase (GST), maltose B binding protein, or protein A are other examples of suitable polypeptide entities.

Depending on the use intended, an anti-HCV-receptor or an anti-envelop antibody of a combination of the invention may be re-engineered so as to optimize stability, solubility, in vivo half-like, or ability to bind additional targets. Genetic engineering approaches as well as chemical modifications to accomplish any or all of these changes in properties are well known in the art. For example, the addition, removal, and/or modification of the constant regions of an antibody are known to play a particularly important role in the bioavailability, distribution, and half-life of therapeutically administered antibodies. The antibody class and subclass, determined by the Fc or constant region of the antibody (which mediates effector functions), when present, imparts important additional properties.

Additional fusion proteins of the invention may be generated through the techniques of DNA shuffling well known in the art (see, for example, U.S. Pat. Nos. 5,605,793; 5,811,238; 5,830,721; 5,834,252; and 5,837,458).

Anti-HCV-receptor and anti-HCV-envelope antibodies of a combination according to the present invention may also be “humanized”: sequence differences between rodent antibodies and human sequences can be minimized by replacing residues which differ from those in the human sequences by site-directed mutagenesis of individual residues or by grafting of entire regions or by chemical synthesis. Humanized antibodies can also be produced using recombinant methods. In the humanized form of the antibody, some, most or all of the amino acids outside the CDR regions are replaced with amino acids from human immunoglobulin molecules, while some, most or all amino acids within one or more CDR regions are unchanged. Small additions, deletions, insertions, substitutions or modifications of amino acids are permissible as long as they do not significantly modify the biological activity of the resulting antibody. Suitable human “replacement” immunoglobulin molecules include IgG1, IgG2, IgG2a, IgG2b, IgG3, IgG4, IgA, IgM, IgD or IgE molecules, and fragments thereof. Alternatively, the T-cell epitopes present in rodent antibodies can be modified by mutation (de-immunization) to generate non-immunogenic rodent antibodies that can be applied for therapeutic purposes in humans (see www.accurobio.com).

Anti-HCV-receptor and anti-HCV-envelope antibodies of a combination according to the invention (or biologically active variants or fragments thereof) may be functionally linked (e.g., by chemical coupling, genetic fusion, non-covalent association or otherwise) to one or more other molecular entities. Methods for the preparation of such modified antibodies (or conjugated antibodies) are known in the art (see, for example, “Affinity Techniques. Enzyme Purification: Part B”, Methods in Enzymol., 1974, Vol. 34, Jakoby and Wilneck (Eds.), Academic Press: New York, N.Y.; and Wilchek and Bayer, Anal. Biochem., 1988, 171: 1-32). Preferably, molecular entities are attached at positions on the antibody molecule that do not interfere with the binding properties of the resulting conjugate, e.g., positions that do not participate in the specific binding of the antibody to its target.

The antibody molecule and the molecular entity may be covalently, directly linked to each other. Or, alternatively, the antibody molecule and the molecular entity may be covalently linked to each other through a linker group. This can be accomplished by using any of a wide variety of stable bifunctional agents well known in the art, including homofunctional and heterofunctional linkers.

In certain embodiments, an antibody of a combination of the present invention (or a biologically active fragment thereof) is conjugated to a therapeutic moiety. Any of a wide variety of therapeutic moieties may be suitable for use in the practice of the present invention including, without limitation, cytotoxins (e.g., cytostatic or cytocidal agents), therapeutic agents, and radioactive metal ions (e.g., alpha-emitters and alpha-emitters attached to macrocyclic chelators such as DOTA). Cytotoxins or cytotoxic agents include any agent that is detrimental to cells. Examples include, but are not limited to, paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, thymidine kinase, endonuclease, RNAse, and puromycin and fragments, variants or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin and doxorubicin), antibiotics (e.g., dactinomycin, bleomycin, mithramycin, and anthramycin), and anti-mitotic agents (e.g., vincristine and vinblastine). The resulting antibody conjugates may find application in the treatment of liver cancer associated with HCV infection (see below).

Other therapeutic moieties include proteins or polypeptides possessing a desired biological activity. Such proteins include, but are not limited to, toxins (e.g., abrin, ricin A, alpha toxin, pseudomonas exotoxin, diphtheria toxin, saporin, momordin, gelonin, pokeweed antiviral protein, alpha-sarcin and cholera toxin); proteins such as tumor necrosis factor, alpha-interferon, beta-interferon, nerve growth factor, platelet derived growth factor, tissue plasminogen activator; apoptotic agents (e.g., TNF-α, TNF-β) or, biological response modifiers (e.g., lymphokines, interleukin-1 (IL-1), interleukin-2 (IL-2), interleukin-6 (IL-6), granulocyte macrophage colony stimulating factor (GM-CSF), granulocyte colony stimulating factor (G-CSF), or other growth factors).

Thus, an inventive combination of the present invention may comprise full length antibodies, biologically active variants or fragments thereof, chimeric antibodies, humanized antibodies, and antibody-derived molecules comprising at least one complementary determining region (CDR) from either a heavy chain or light chain variable region of an anti-HCV-receptor antibody or anti-HCV-envelope antibody, including molecules such as Fab fragments, F(ab′)₂ fragments, Fd fragments, Fabc fragments, Sc antibodies (single chain antibodies), diabodies, individual antibody light single chains, individual antibody heavy chains, chimeric fusions between antibody chains and other molecules, and antibody conjugates, such as antibodies conjugated to a therapeutic agent.

### D. Properties of the Combinations

A combination according to the present invention is such that (1) it is intended for use in the treatment or the prevention of HCV infection and (2) the at least one anti-HCV-envelope antibody and at least one anti-HCV-receptor antibody act in a highly synergistic manner on the inhibition of HCV infection.

In certain embodiments, the anti-HCV-envelope antibody decreases the IC₅₀ for the inhibition of HCV infection by the anti-HCV-receptor antibody by a factor of up to at least 10 fold or up to at least 25 fold, preferably up to at least 50 fold, more preferably up to at least 75 fold, and even more preferably up to 100 fold. In other words, in the presence of the anti-HCV-envelope antibody, the concentration of anti-HCV-receptor antibody necessary to obtain a 50% inhibition of HCV entry is at least 10 times, at least 25 times, preferably at least 50 times, more preferably at least 75 times, and even more preferably more than 100 times lower than the concentration of the anti-HCV-receptor antibody that would be necessary to obtain the same HCV entry inhibition in the absence of anti-HCV-envelope antibody.

In other embodiments, the anti-HCV-receptor antibody decreases the IC₅₀ for the inhibition of HCV infection by the anti-HCV-envelope antibody by a factor of up to at least 10 fold or up to at least 25 fold, preferably up to at least 50 fold, more preferably up to at least 75 fold, and even more preferably up to 100 fold. In other words, in the presence of the anti-HCV-receptor antibody, the concentration of anti-HCV-envelope antibody necessary to obtain a 50% inhibition of HCV entry is at least 10 times, at least 25 times, preferably at least 50 times, more preferably at least 75 times, and even more preferably more than 100 times lower than the concentration of the anti-HCV-envelope antibody that would be necessary to obtain the same HCV entry inhibition in the absence of anti-HCV-receptor antibody.

In certain embodiments, a combination of the present invention is characterized by a combination index (CI) that is lower than 1 (which is defined as a marked synergy). A combination of the present invention is preferably characterized by a CI lower than 0.75, more preferably by a CI lower than 0.50, and even more preferably by a CI lower than 0.30.

### II—Treatment or Prevention of HCV Infection and HCV-Associated Diseases

### A. Indications

The combinations of antibodies according of the present invention may be used in therapeutic and prophylactic methods to treat and/or prevent HCV infection, or to treat and/or prevent a liver disease or a pathological condition affecting HCV-susceptible cells, such as liver cells, lymphoid cells, or monocytes/macrophages.

Methods of treatment of the present invention may be accomplished using an inventive combination or a pharmaceutical composition comprising an inventive combination (see below). These methods generally comprise administration of an effective amount of at least one anti-HCV-envelope antibody and at least one anti-HCV-receptor antibody (as defined above), or a pharmaceutical composition thereof, to a subject in need thereof. The anti-HCV-envelope antibody and the anti-HCV-receptor antibody may be administered concurrently (i.e., together or separately but at about the same time, e.g., within 5 minutes, 15 minutes or 30 minutes of each other), or alternatively, they may be administered sequentially (i.e., separately and at different times, e.g., different times of the same day or different times of the same week or different times of the same month, etc. . . . ).

Administration may be performed using any of the methods known to one skilled in the art. In particular, the antibodies or composition may be administered by various routes including, but not limited to, aerosol, parenteral, oral or topical route.

In general, the combination of antibodies or composition thereof will be administered in an effective amount, i.e. an amount that is sufficient to fulfill its intended purpose. The exact amount of antibodies or pharmaceutical composition to be administered will vary from subject to subject, depending on the age, sex, weight and general health condition of the subject to be treated, the desired biological or medical response (e.g., prevention of HCV infection or treatment of HCV-associated liver disease), and the like. In many embodiments, an effective amount is one that inhibits or prevents HCV from entering into a subject's susceptible cells and/or infecting a subject's cells, so as to prevent HCV infection, treat or prevent liver disease or another HCV-associated pathology in the subject.

Antibodies and compositions of the present invention may be used in a variety of therapeutic or prophylactic methods. In particular, the present invention provides a method for treating or preventing a liver disease or pathology in a subject, which comprises administering to the subject an effective amount of at least one anti-HCV-envelope antibody and at least one anti-HCV-receptor antibody (as defined above) (or composition thereof) which inhibits HCV from entering or infecting the subject's cells, so as to treat or prevent the liver disease or pathology in the subject. The liver disease or pathology may be inflammation of the liver, liver fibrosis, cirrhosis, and/or hepatocellular carcinoma (i.e., liver cancer) associated with HCV infection.

The present invention also provides a method for treating or preventing a HCV-associated disease or condition (including a liver disease) in a subject, which comprises administering to the subject an effective amount of at least one anti-HCV-envelope antibody and at least one anti-HCV-receptor antibody (as defined above) (or composition thereof) which inhibits HCV from entering or infecting the subject's cells, so as to treat or prevent the HCV-associated disease or condition in the subject. In certain embodiments of the present invention, the antibodies (or composition thereof) are administered to a subject diagnosed with acute hepatitis C. In other embodiments of the invention, the antibodies (or composition thereof) are administered to a subject diagnosed with chronic hepatitis C.

Administration of an inventive composition according to such methods may result in amelioration of at least one of the symptoms experienced by the individual including, but not limited to, symptoms of acute hepatitis C such as decreased appetite, fatigue, abdominal pain, jaundice, itching, and flu-like symptoms; symptoms of chronic hepatitis C such as fatigue, marked weight loss, flu-like symptoms, muscle pain, joint pain, intermittent low-grade fevers, itching, sleep disturbances, abdominal pain, appetite changes, nausea, diarrhea, dyspepsia, cognitive changes, depression, headaches, and mood swings; symptoms of cirrhosis such as ascites, bruising and bleeding tendency, bone pain, varices (especially in the stomach and esophagus), steatorrhea, jaundice and hepatic encephalopathy; and symptoms of extrahepatic manifestations associated with HCV such as thyroiditis, porphyria cutanea tarda, cryoglobulinemia, glomerulonephritis, sicca syndrome, thrombocytopenia, lichen planus, diabetes mellitus and B-cell lymphoproliferative disorders.

Alternatively or additionally, administration of antibodies or composition thereof according to such methods may slow down, reduce, stop or alleviate the progression of HCV infection or an HCV-associated disease, or reverse the progression to the point of eliminating the infection or disease. Administration of antibodies or composition thereof according to such methods may also result in a reduction in the number of viral infections, reduction in the number of infectious viral particles, and/or reduction in the number of virally infected cells.

The effects of a treatment according to the invention may be monitored using any of the assays known in the art for the diagnosis of HCV infection and/or liver disease. Such assays include, but are not limited to, serological blood tests, liver function tests to measure one or more of albumin, alanine transaminase (ALT), alkaline phosphatase (ALP), aspartate transaminase (AST), and gamma glutamyl transpeptidase (GGT), and molecular nucleic acid tests using different techniques such as polymerase chain reaction (PCR), transcription mediated amplification (TMA), or branched DNA (bDNA).

Antibodies and compositions of the present invention may also be used in immunization therapies. Accordingly, the present invention provides a method of reducing the likelihood of susceptible cells of becoming infected with HCV as a result of contact with HCV. The method comprises contacting the susceptible cells with an effective amount of at least one anti-HCV-envelope antibody and at least one anti-HCV-receptor antibody (as defined above) or a composition thereof which inhibits HCV from entering or infecting the susceptible cells, so as to reduce the likelihood of the cells to become infected with HCV as a result of contact with HCV. The present invention also provides a method of reducing the likelihood of a subject's susceptible cells of becoming infected with HCV as a result of contact with HCV. In this method, contacting the susceptible cells with the antibodies or composition may be performed by administrating the antibodies or a composition thereof to the subject.

Reducing the likelihood of susceptible cells or of a subject of becoming infected with HCV means decreasing the probability of susceptible cells or a subject to become infected with HCV as a result of contact with HCV. The decrease may be of any significant amount, e.g., at least a 2-fold decrease, more than a 2-fold decrease, at least a 10-fold decrease, more than a 10-fold decrease, at least a 100-fold decrease, or more than a 100-fold decrease.

In certain embodiments, the subject is infected with HCV prior to administration of the inventive antibody or composition. In other embodiments, the subject is not infected with HCV prior to administration of the inventive antibody or composition. In yet other embodiments, the subject is not infected with, but has been exposed to, HCV. In certain embodiments, the subject may be infected with HIV or HBV.

For example, the methods of the present invention may be used to reduce the likelihood of a subject's susceptible cells of becoming infected with HCV as a result of liver transplant. As already mentioned above, when a diseased liver is removed from a HCV-infected patient, serum viral levels plummet. However, after receiving a healthy liver transplant, virus levels rebound and can surpass pre-transplant levels within a few days (Powers et al., Liver Transpl., 2006, 12: 207-216). Liver transplant patients may benefit from administration of a combination of antibodies according the invention. Administration may be performed prior to liver transplant, during liver transplant, and/or following liver transplant.

Other subjects that may benefit from administration of a combination of antibodies according to the present invention include, but are not limited to, babies born to HCV-infected mothers, in particular if the mother is also HIV-positive; health-care workers who have been in contact with HCV-contaminated blood or blood contaminated medical instruments; drug users who have been exposed to HCV by sharing equipments for injecting or otherwise administering drugs; and people who have been exposed to HCV through tattooing, ear/body piercing and acupuncture with poor infection control procedures.

Other subjects that may benefit from administration of a combination of antibodies according to the invention include, but are not limited to, subjects that exhibit one or more factors that are known to increase the rate of HCV disease progression. Such factors include, in particular, age, gender (males generally exhibit more rapid disease progression than females), alcohol consumption, HIV co-infection (associated with a markedly increased rate of disease progression), and fatty liver.

In certain embodiments, a combination of antibodies or a composition thereof is administered alone according to a method of treatment of the present invention. In other embodiments, a combination of antibodies or a composition thereof is administered in combination with at least one additional therapeutic agent. The combination or composition may be administered prior to administration of the therapeutic agent, concurrently with the therapeutic agent, and/or following administration of the therapeutic agent.

Therapeutic agents that may be administered in combination with an inventive combination or composition may be selected among a large variety of biologically active compounds that are known to have a beneficial effect in the treatment or prevention of HCV infection, or a HCV-associated disease or condition. Such agents include, in particular, antiviral agents including, but not limited to, interferons (e.g., interferon-alpha, pegylated interferon-alpha), ribavirin, anti-HCV (monoclonal or polyclonal) antibodies, RNA polymerase inhibitors, protease inhibitors, IRES inhibitors, helicase inhibitors, antisense compounds, ribozymes, and any combination thereof.

### B. Administration

An inventive combination of antibodies, (optionally after formulation with one or more appropriate pharmaceutically acceptable carriers or excipients), in a desired dosage can be administered to a subject in need thereof by any suitable route. Various delivery systems are known and can be used to administer antibodies of the present invention, including tablets, capsules, injectable solutions, encapsulation in liposomes, microparticles, microcapsules, etc. Methods of administration include, but are not limited to, dermal, intradermal, intramuscular, intraperitoneal, intralesional, intravenous, subcutaneous, intranasal, pulmonary, epidural, ocular, and oral routes. An inventive combination or composition may be administered by any convenient or other appropriate route, for example, by infusion or bolus injection, by absorption through epithelial or mucocutaneous linings (e.g., oral, mucosa, rectal and intestinal mucosa, etc). Administration can be systemic or local. Parenteral administration may be preferentially directed to the patient's liver, such as by catheterization to hepatic arteries or into a bile duct. As will be appreciated by those of ordinary skill in the art, in embodiments where an inventive combination of antibodies is administered along with an additional therapeutic agent, the antibodies and therapeutic agent may be administered by the same route (e.g., intravenously) or by different routes (e.g., intravenously and orally).

### C. Dosage

Administration of an inventive combination of antibodies (or a composition thereof) of the present invention will be in a dosage such that the amount delivered is effective for the intended purpose. The route of administration, formulation and dosage administered will depend upon the therapeutic effect desired, the severity of the HCV-related condition to be treated if already present, the presence of any infection, the age, sex, weight, and general health condition of the patient as well as upon the potency, bioavailability, and in vivo half-life of the antibodies used, the use (or not) of concomitant therapies, and other clinical factors. These factors are readily determinable by the attending physician in the course of the therapy. Alternatively or additionally, the dosage to be administered can be determined from studies using animal models (e.g., chimpanzee or mice). Adjusting the dose to achieve maximal efficacy based on these or other methods are well known in the art and are within the capabilities of trained physicians. As studies are conducted using the inventive combination of monoclonal antibodies, further information will emerge regarding the appropriate dosage levels and duration of treatment.

A treatment according to the present invention may consist of a single dose or multiple doses. Thus, administration of an inventive combination of antibodies, or composition thereof, may be constant for a certain period of time or periodic and at specific intervals, e.g., hourly, daily, weekly (or at some other multiple day interval), monthly, yearly (e.g., in a time release form). Alternatively, the delivery may occur at multiple times during a given time period, e.g., two or more times per week; two or more times per month, and the like. The delivery may be continuous delivery for a period of time, e.g., intravenous delivery.

In general, the amount of antibodies administered will preferably be in the range of about 1 ng/kg to about 100 mg/kg body weight of the subject, for example, between about 100 ng/kg and about 50 mg/kg body weight of the subject; or between about 1 μg/kg and about 10 mg/kg body weight of the subject, or between about 100 μg/kg and about 1 mg/kg body weight of the subject.

### III—Pharmaceutical Compositions

As mentioned above, the combination of antibodies of the invention may be administered per se or as a pharmaceutical composition. Accordingly, the present invention provides pharmaceutical compositions comprising an effective amount of at least one anti-HCV-envelope antibody and at least one anti-HCV-receptor antibody as described herein and at least one pharmaceutically acceptable carrier or excipient. In some embodiments, the composition further comprises one or more additional biologically active agents.

The combination of antibodies and pharmaceutical compositions thereof may be administered in any amount and using any route of administration effective for achieving the desired prophylactic and/or therapeutic effect. The optimal pharmaceutical formulation can be varied depending upon the route of administration and desired dosage. Such formulations may influence the physical state, stability, rate of in vivo release, and rate of in vivo clearance of the administered active ingredient.

The pharmaceutical compositions of the present invention may be formulated in dosage unit form for ease of administration and uniformity of dosage. The expression “unit dosage form”, as used herein, refers to a physically discrete unit of an anti-HCV-envelope antibody or of an anti-HCV-receptor antibody or of both an anti-HCV-envelope antibody and an anti-HCV-receptor antibody for the patient to be treated. It will be understood, however, that the total daily dosage of the compositions will be decided by the attending physician within the scope of sound medical judgement.

### A. Formulation

Injectable preparations, for example, sterile injectable aqueous or oleaginous suspensions may be formulated according to the known art using suitable dispersing or wetting agents, and suspending agents. The sterile injectable preparation may also be a sterile injectable solution, suspension or emulsion in a non-toxic parenterally acceptable diluent or solvent, for example, as a solution in 2,3-butanediol. Among the acceptable vehicles and solvents that may be employed are water, Ringer's solution, U.S.P. and isotonic sodium chloride solution. In addition, sterile, fixed oils are conventionally employed as a solution or suspending medium. For this purpose any bland fixed oil can be employed including synthetic mono- or di-glycerides. Fatty acids such as oleic acid may also be used in the preparation of injectable formulations. Sterile liquid carriers are useful in sterile liquid form compositions for parenteral administration.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Liquid pharmaceutical compositions which are sterile solutions or suspensions can be administered by, for example, intravenous, intramuscular, intraperitoneal or subcutaneous injection. Injection may be via single push or by gradual infusion. Where necessary or desired, the composition may include a local anesthetic to ease pain at the site of injection.

In order to prolong the effect of an active ingredient (here a combination of an anti-HCV-envelope antibody and an anti-HCV-receptor antibody), it is often desirable to slow the absorption of the ingredient from subcutaneous or intramuscular injection. Delaying absorption of a parenterally administered active ingredient may be accomplished by dissolving or suspending the ingredient in an oil vehicle. Injectable depot forms are made by forming micro-encapsulated matrices of the active ingredient in biodegradable polymers such as polylactide-polyglycolide. Depending upon the ratio of active ingredient to polymer and the nature of the particular polymer employed, the rate of ingredient release can be controlled. Examples of other biodegradable polymers include poly(orthoesters) and poly(anhydrides). Depot injectable formulations can also be prepared by entrapping the active ingredient in liposomes or microemulsions which are compatible with body tissues.

Liquid dosage forms for oral administration include, but are not limited to, pharmaceutically acceptable emulsions, microemulsions, solutions, suspensions, syrups, elixirs, and pressurized compositions. In addition to the antibodies, the liquid dosage form may contain inert diluents commonly used in the art such as, for example, water or other solvent, solubilising agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cotton seed, ground nut, corn, germ, olive, castor, and sesame oils), glycerol, tetrahydrofurfuryl alcohol, polyethylene glycols, and fatty acid esters of sorbitan and mixtures thereof. Besides inert diluents, the oral compositions can also include adjuvants such as wetting agents, suspending agents, preservatives, sweetening, flavouring, and perfuming agents, thickening agents, colors, viscosity regulators, stabilizes or osmo-regulators. Examples of suitable liquid carriers for oral administration include water (potentially containing additives as above, e.g., cellulose derivatives, such as sodium carboxymethyl cellulose solution), alcohols (including monohydric alcohols and polyhydric alcohols such as glycols) and their derivatives, and oils (e.g., fractionated coconut oil and arachis oil). For pressurized compositions, the liquid carrier can be halogenated hydrocarbon or other pharmaceutically acceptable propellant.

Solid dosage forms for oral administration include, for example, capsules, tablets, pills, powders, and granules. In such solid dosage forms, an inventive combination of antibodies may be mixed with at least one inert, physiologically acceptable excipient or carrier such as sodium citrate or dicalcium phosphate and one or more of: (a) fillers or extenders such as starches, lactose, sucrose, glucose, mannital, and silicic acid; (b) binders such as, for example, carboxymethylcellulose, alginates, gelatine, polyvinylpyrrolidone, sucrose, and acacia; (c) humectants such as glycerol; (d) disintegrating agents such as agar-agar, calcium carbonate, potato or tapioca starch, alginic acid, certain silicates, and sodium carbonate; (e) solution retarding agents such as paraffin; absorption accelerators such as quaternary ammonium compounds; (g) wetting agents such as, for example, cetyl alcohol and glycerol monostearate; (h) absorbents such as kaolin and bentonite clay; and (i) lubricants such as talc, calcium stearate, magnesium stearate, solid polyethylene glycols, sodium lauryl sulphate, and mixtures thereof. Other excipients suitable for solid formulations include surface modifying agents such as non-ionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine. In the case of capsules, tablets and pills, the dosage form may also comprise buffering agents.

Solid compositions of a similar type may also be employed as fillers in soft and hard-filled gelatine capsules using such excipients as lactose or milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. They may optionally contain opacifying agents and can also be of a composition such that they release the active ingredient(s) only, or preferably, in a certain part of the intestinal tract, optionally, in a delaying manner. Examples of embedding compositions which can be used include polymeric substances and waxes.

In certain embodiments, it may be desirable to administer an inventive composition locally to an area in need of treatment (e.g., the liver). This may be achieved, for example, and not by way of limitation, by local infusion during surgery (e.g., liver transplant), topical application, by injection, by means of a catheter, by means of suppository, or by means of a skin patch or stent or other implant.

For topical administration, the composition is preferably formulated as a gel, an ointment, a lotion, or a cream which can include carriers such as water, glycerol, alcohol, propylene glycol, fatty alcohols, triglycerides, fatty acid esters, or mineral oil. Other topical carriers include liquid petroleum, isopropyl palmitate, polyethylene glycol, ethanol (95%), polyoxyethylenemonolaurat (5%) in water, or sodium lauryl sulphate (5%) in water. Other materials such as antioxidants, humectants, viscosity stabilizers, and similar agents may be added as necessary.

In addition, in certain instances, it is expected that the inventive compositions may be disposed within transdermal devices placed upon, in, or under the skin. Such devices include patches, implants, and injections which release the active ingredient by either passive or active release mechanisms. Transdermal administrations include all administration across the surface of the body and the inner linings of bodily passage including epithelial and mucosal tissues. Such administrations may be carried out using the present compositions in lotions, creams, foams, patches, suspensions, solutions, and suppositories (rectal and vaginal).

Transdermal administration may be accomplished through the use of a transdermal patch containing an active ingredient (i.e., a combination of an anti-HCV-envelope antibody and of an anti-HCV-receptor antibody) and a carrier that is non-toxic to the skin, and allows the delivery of the ingredient for systemic absorption into the bloodstream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may be suitable. A variety of occlusive devices may be used to release the active ingredient into the bloodstream such as a semi-permeable membrane covering a reservoir containing the active ingredient with or without a carrier, or a matrix containing the active ingredient.

Suppository formulations may be made from traditional materials, including cocoa butter, with or without the addition of waxes to alter the suppository's melting point, and glycerine. Water soluble suppository bases, such as polyethylene glycols of various molecular weights, may also be used.

When a pharmaceutical composition of the present invention is used as “vaccine” to prevent HCV-susceptible cells to become infected with HCV, the pharmaceutical composition may further comprise vaccine carriers known in the art such as, for example, thyroglobulin, albumin, tetanus toxoid, and polyamino acids such as polymers of D-lysine and D-glutamate. The vaccine may also include any of a variety of well known adjuvants such as, for example, incomplete Freund's adjuvant, alum, aluminium phosphate, aluminium hydroxide, monophosphoryl lipid A (MPL, GlaxoSmithKline), a saponin, CpG oligonucleotides, montanide, vitamin A and various water-in-oil emulsions prepared from biodegradable oils such as squalene and/or tocopherol, Quil A, Ribi Detox, CRL-1005, L-121 and combinations thereof.

Materials and methods for producing various formulations are known in the art and may be adapted for practicing the subject invention. Suitable formulations for the delivery of antibodies can be found, for example, in “Remington's Pharmaceutical Sciences”, E. W. Martin, 18^{th} Ed., 1990, Mack Publishing Co.: Easton, Pa.

### B. Additional Biologically Active Agents

In certain embodiments, an inventive combination of antibodies is the only active ingredient in a pharmaceutical composition of the present invention. In other embodiments, the pharmaceutical composition further comprises one or more biologically active agents. Examples of suitable biologically active agents include, but are not limited to, vaccine adjuvants and therapeutic agents such as anti-viral agents (as described above), anti-inflammatory agents, immunomodulatory agents, analgesics, antimicrobial agents, antibacterial agents, antibiotics, antioxidants, antiseptic agents, and combinations thereof.

In such pharmaceutical compositions, antibodies and additional therapeutic agent(s) may be combined in one or more preparations for simultaneous, separate or sequential administration of the antibodies and therapeutic agent(s). More specifically, an inventive composition may be formulated in such a way that the antibodies and therapeutic agent(s) can be administered together or independently from each other. For example, an anti-HCV-envelope antibody, an anti-HCV-receptor antibody and a therapeutic agent can be formulated together in a single composition. Alternatively, they may be maintained (e.g., in different compositions and/or containers) and administered separately.

### C. Pharmaceutical Packs of Kits

In another aspect, the present invention provides a pharmaceutical pack or kit comprising one or more containers (e.g., vials, ampoules, test tubes, flasks or bottles) containing one or more ingredients of an inventive pharmaceutical composition, allowing administration of a combination of antibodies of the present invention.

Different ingredients of a pharmaceutical pack or kit may be supplied in a solid (e.g., lyophilized) or liquid form. Each ingredient will generally be suitable as aliquoted in its respective container or provided in a concentrated form. Pharmaceutical packs or kits may include media for the reconstitution of lyophilized ingredients. Individual containers of the kits will preferably be maintained in close confinement for commercial sale.

In certain embodiments, a pharmaceutical pack or kit includes one or more additional therapeutic agent(s) (e.g., one or more anti-viral agents, as described above). Optionally associated with the container(s) can be a notice or package insert in the form prescribed by a governmental agency regulating the manufacture, use or sale of pharmaceutical or biological products, which notice reflects approval by the agency of manufacture, use or sale for human administration. The notice of package insert may contain instructions for use of a pharmaceutical composition according to methods of treatment disclosed herein.

An identifier, e.g., a bar code, radio frequency, ID tags, etc., may be present in or on the kit. The identifier can be used, for example, to uniquely identify the kit for purposes of quality control, inventory control, tracking movement between workstations, etc.

### EXAMPLES

The following example describes some of the preferred modes of making and practicing the present invention. However, it should be understood that the examples are for illustrative purposes only and are not meant to limit the scope of the invention. Furthermore, unless the description in an Example is presented in the past tense, the text, like the rest of the specification, is not intended to suggest that experiments were actually performed or data are actually obtained.

Some of the results reported below are presented in I. Fofana et al., “Monoclonal anti-Claudin 1 Antibodies Prevent Hepatitis C Virus Infection of Primary Human Hepatocytes”, which has been published in Gastroenterology (2010, 139: 953-964).

### Example 1

### Inhibition of HCVpp Infection Using Combinations of Antibodies

### Materials and Methods

Primary Hepatocytes and Cell Lines.

Culture of primary human hepatocytes (PHH) (Krieger et al., Hepatology, 2010, 51: 1144-1157), Huh7.5.1 (Zhong et al., Proc. Natl. Acad. Sci. USA, 2005, 102: 9294-2929), and CHO (Krieger et al., Hepatology, 2010, 51: 1144-1157) cells, which have previously been described, were used in this study.

Production of Anti-CLDN1 mAbs.

Anti-CLDN1 mAbs were raised by genetic immunization of Wistar rats using an eukaryotic expression vector encoding the full-length human CLDN1 cDNA as described in EP 08 305 597 and WO 2010/034812. Following completion of immunization, antibodies were selected by flow cytometry for their ability to bind to human CLDN1 expressed on the cell surface of non-permeabilized HEK293T-BOSC23 cells and CHO cells which had been transfected with pCMV-SPORT6/CLDN1. In the present invention, anti-CLDN1 mAb OM-7D3-B3 was used.

Other Monoclonal Antibodies.

Anti-E1 and anti-E2 mAbs were purchased from Innogenetics. Anti-HCV IgG was produced from HCV-infected patients. Anti-SR-BI and anti-CD81 monoclonal antibodies have been produced by DNA immunization in rodents in a similar manner as described for anti-CLDN1 (Fofana et al., Gastroenterology, 2010, 139: 953-964).

HCVpp Production and Infection.

HCVpp (strains P02VJ and P04VD) were produced as previously described (Fafi-Kremer et al., J. Exp. Med., 2010). Patient-derived HCVpp were produced from 6 patients (P01-P06) undergoing liver transplantation using full-length E1E2 expression constructs generated from circulating HCV as previously described (Pestka et al., Proc. Natl. Acad. Sci. USA, 2007, 104: 6025-6030; 24). Huh7.5.1, Huh7 cells or PHH were pre-incubated with antibodies for 1 hour and incubated for 4 hours at 37° C. with HCVpp. Viral infection was analyzed as described (Krieger et al., Hepatology, 2010, 51: 1144-1157; Koutsoudakis et al., J. Virol., 2006, 80: 5308-5320). For antibody-mediated neutralization, HCVpp were pre-incubated with autologous anti-HCV serum (Fifa-Kremer, in revision 2010), anti-E2 mAb (IGH461, Innogenetics) (Haberstroh et al., Gastroenterology, 2008, 135: 1719-1728) and anti-HCV IgG purified from a chronically infected patient as previously described (Haberstroh et al., Gastroenterology, 2008, 135: 1719-1728; von Hahn et al., Gastroenterology, 2007, 132: 667-378).

Toxicity Assays.

Cytotoxic effects on cells were assessed by analyzing the ability to metabolize 3-(4,5-dimethylthiazol-2-yl)-2,5-diphenyltetrazolium bromide (MTT) as previously described (Mosmann et al., J. Immunol. Methods, 1983, 65: 55-63).

Statistical Analysis.

Results are expressed as means±standard deviation (SD). Statistical analyses were performed using Student's t test with a P value of <0.05 being considered statistically significant.

### Results and Discussion

To investigate whether the concomitant administration of anti-HCV-receptor mAbs with anti-HCV-envelope antibodies results in an additive effect in the inhibition of viral infection, the Applicants have pre-incubated patient-derived HCVpp either with monoclonal anti-E2 antibody IGH461, anti-E1 antibody IGH526 or with purified heterologous anti-HCV IgG obtained from a chronically infected patient and studied their ability to inhibit patient-derived HCV pseudoparticles infection in cells pre-incubated with each of the following anti-HCV-receptor monoclonal antibodies: anti-CLDN1 mAb (OM-7D3-B3), anti-SR-BI mAb (NK-8H5-E3), and anti-CD81 mAb (QV-6A8-F2CA).

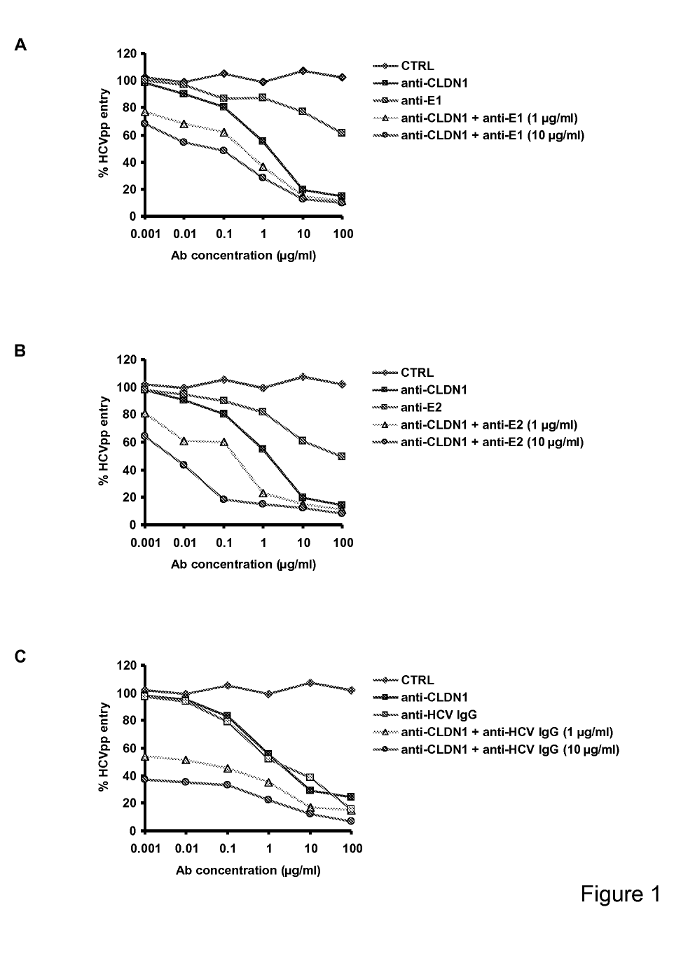

The results obtained for anti-CLDN1 mAbs are presented on FIG. 1. Interestingly, pre-incubation of infectious viral particles with either cross-neutralizing anti-E2 mAb, anti-E1 mAb or purified heterologous anti-HCV IgG resulted in a marked synergistic effect of inhibition of HCV infection, decreasing the IC₅₀ of anti-CLDN1 up to 100 fold.

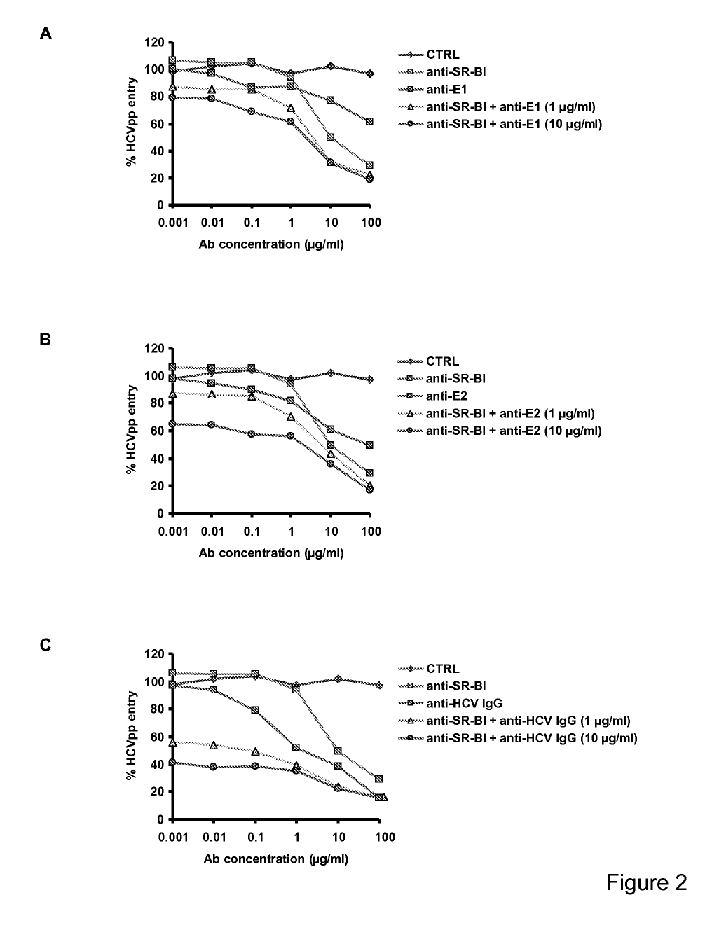

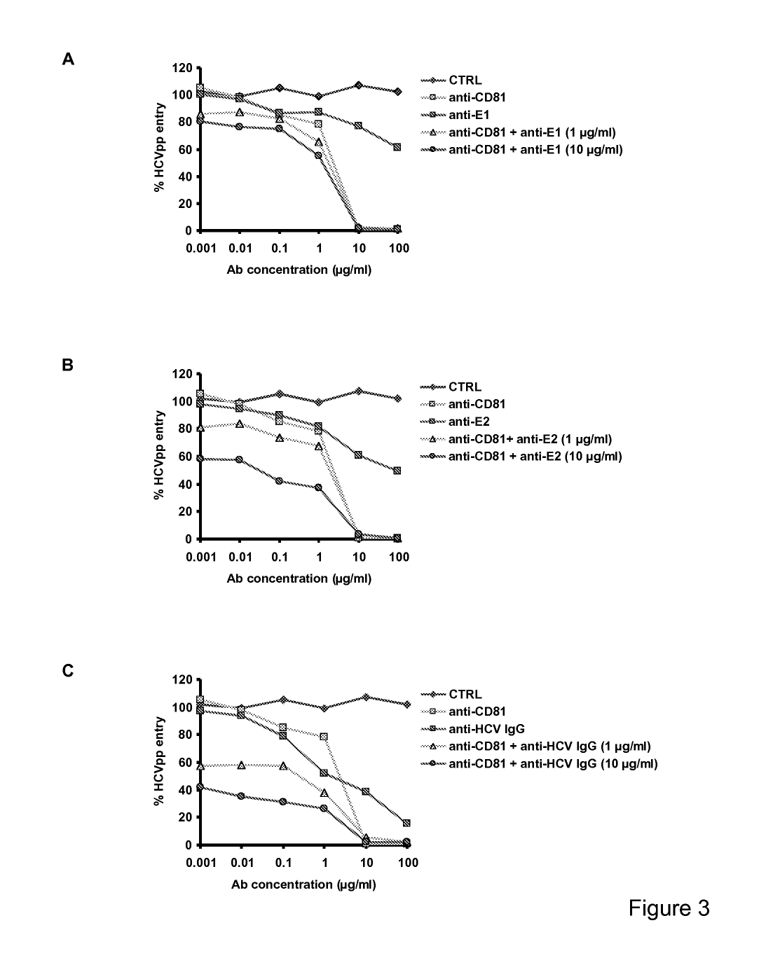

The results obtained for anti-SR-BI mAbs and anti-CD81 mAbs are presented on FIG. 2 and FIG. 3, respectively.

In all the cases, synergy was further confirmed by calculating the combination index CI as previously described (Zhao et al., Clin. Cancer Res., 2004, 10: 7994-8004) determined in a series of side-by-side experiments. The results obtained are presented in the table below. A CI of less than 1 indicates synergy, while a CI equal to 1 indicates additivity and a CI or more than 1 indicates antagonism.

A very pronounced effect was observed for combinations of envelope-specific monoclonal antibodies and anti-CLDN1 antibodies. This marked synergy observed for this combination may be due to the fact that envelope- and CLDN1-specific antibodies target different and complementary targets during entry. Indeed, whereas envelope-specific antibodies have been shown to bind to E2 or E1/E2 complexes interacting with CD81 and SR-BI (for review see Zeisel et al., J. Hepatol., 2011, 54: 566-576), the CLDN1-specific antibody has been shown to interact with CLDN1 resulting in the impairment of CD81-CLDN1 interactions (Krieger et al., Hepatology, 2010, 51: 1144-1157). The fact that one of the most effective combination comprised the combination of envelope- and CLDN1-specific antibodies may also have important clinical and therapeutic implications since it suggests that this combination may be of key interest for the further preclinical and clinical development of antiviral strategies.

TABLE 1
IC₅₀ and CI for each of the combination studied on HCVpp infection.

| anti-HCV-envelope | anti-HCV- | IC₅₀ | |
|---|---|---|---|
| antibody | receptor antibody | (μg/mL) | CI |
| isotype control | anti-CLDN1 | 1.5 | |
| anti-E1 mAb | anti-CLDN1 | 0.3 | 0.20 |
| anti-E2 mAb | anti-CLDN1 | 0.2 | 0.26 |
| anti-HCV IgG | anti-CLDN1 | 0.02 | 0.51 |
| isotype control | anti-SRBI | 10 | |
| anti-El mAb | anti-SRBI | 4 | 0.40 |
| anti-E2 mAb | anti-SRBI | 4 | 0.40 |
| anti-HCV IgG | anti-SRBI | 0.07 | 0.50 |
| isotype control | anti-CD81 | 2.5 | |
| anti-E1 mAb | anti-CD81 | 2 | 0.80 |
| anti-E2 mAb | anti-CD81 | 2 | 0.81 |
| anti-HCV IgG | anti-CD81 | 0.3 | 0.62 |

With these results, the present Applicants have shown for the first time that a cross-neutralizing anti-HCV-envelope antibody or a purified heterologous anti-HCV IgG and anti-HCV-receptor antibodies (anti-CLDN1, anti-SR-BI and anti-CD81 mAbs) act in a highly synergistic manner on the inhibition of entry of highly infectious HCV escape variants and applied for treatment of chronic HCV infection.

These results demonstrate that the combination of anti-HCV receptor antibodies with cross-neutralizing anti-HCV-envelope antibodies is a highly original and effective antiviral approach to prevent primary HCV infection, such as, for example, after liver transplantation. This approach may also restrain virus spread in chronically infected patients.

In order to address the relevance of these findings for other genotypes, the effects of combinations comprising an anti-HCV-envelope antibody and an anti-CLDN1 antibody were also studied against infection of HCVpp expressing envelope glycoproteins of genotypes 1-6. The results obtained are presented in Table 2.

TABLE 2
IC₅₀ and CI for each of the combination studied on HCVpp
infection from all major genotypes.

| | | IC₅₀ | |
|---|---|---|---|
| Genotype | Compound or Combination | (μg/mL) | CI |
| la | anti-CLDN1 | 6.0 | |
| | anti-CLDN1 + anti-E1 mAb (1 μg/mL) | 0.02 | 0.05 |
| | anti-CLDN1 + anti-E2 mAb (1 μg/mL) | 2 | 0.34 |
| | anti-CLDN1 + anti-HCV IgG (1 μg/mL) | 0.003 | 0.16 |
| 1b | anti-CLDN1 | 4.0 | |
| | anti-CLDN1 + anti-E1 mAb (1 μg/mL) | 0.4 | 0.12 |
| | anti-CLDN1 + anti-E2 mAb (1 μg/mL) | 0.03 | 0.06 |
| | anti-CLDN1 + anti-HCV IgG (1 μg/mL) | 0.006 | 0.40 |
| 2a | anti-CLDN1 | 3.0 | |
| | anti-CLDN1 + anti-E1 mAb (1 μg/mL) | 0.03 | 0.05 |
| | anti-CLDN1 + anti-E2 mAb (1 μg/mL) | 0.8 | 0.27 |
| | anti-CLDN1 + anti-HCV IgG (1 μg/mL) | 0.005 | 0.22 |
| 3a | anti-CLDN1 | 0.07 | |
| | anti-CLDN1 + anti-E1 mAb (1 μg/mL) | 0.04 | 0.57 |
| | anti-CLDN1 + anti-E2 mAb (1 μg/mL) | 0.03 | 0.43 |
| | anti-CLDN1 + anti-HCV IgG (1 μg/mL) | 0.001 | 0.41 |
| 4 | anti-CLDN1 | 0.3 | |
| | anti-CLDN1 + anti-E1 mAb (1 μg/mL) | 0.05 | 0.33 |
| | anti-CLDN1 + anti-E2 mAb (1 μg/mL) | 0.1 | 0.35 |
| | anti-CLDN1 + anti-HCV IgG (1 μg/mL) | 0.001 | 0.30 |
| 5 | anti-CLDN1 | 0.8 | |
| | anti-CLDN1 + anti-E1 mAb (1 μg/mL) | 0.02 | 0.31 |
| | anti-CLDN1 + anti-E2 mAb (1 μg/mL) | 0.001 | 0.40 |
| | anti-CLDN1 + anti-HCV IgG (1 μg/mL) | 0.001 | 0.35 |
| 6 | anti-CLDN1 | 5 | |
| | anti-CLDN1 + anti-E1 mAb (1 μg/mL) | 0.4 | 0.08 |
| | anti-CLDN1 + anti-E2 mAb (1 μg/mL) | 2 | 0.40 |
| | anti-CLDN1 + anti-HCV IgG (1 μg/mL) | 0.001 | 0.33 |

The results obtained show that the use of a combination according to the invention resulted in CIs of 0.05-057, indicative of a synergy on the inhibition of entry of HCVpp bearing envelope glycoproteins from all major HCV genotypes.

These results show, for the first time, that a cross-neutralizing anti-HCV-envelope antibody or a purified heterologous anti-HCV IgG and anti-HCV-receptor antibodies (anti-CLDN1, anti-SR-BI, and anti-CD81 mAbs) act in a highly synergistic manner on the inhibition of entry of HCV of all major genotypes.

### Example 2

### Inhibition of HCVcc Infection Using Combinations of Antibodies

Cell culture derived HCVcc (Luc-Jc1, genotype 2a) (Krieger et al., Hepatology, 2010, 54: 1144-1157) were preincubated with purified heterologous anti-HCV IgG (1 or 10 μg/mL) obtained from an unrelated chronically infected subject or isotype control IgG for 1 hour at 37° C. and added to Huh7 cells pre-incubated with increasing concentrations of anti-CLDN1 OM-7D3-B3, anti-SR-BI NK-8H5-E3, anti-CD81 QV-6A8-F2C4, rat or mouse isotype control mAbs. HCVcc infection was analyzed by luciferase reporter gene expression as previously described (Krieger et al., Hepatology, 2010, 54: 1144-1157; Fofana et al., Gastroenterology, 2010, 139: 953-964). Combination of anti-HCV-IgG and anti-HCV-receptor antibody resulted in a synergistic effect with a CI of 0.63 (for 1 μg/mL of anti-HCV IgG and anti-CLDN1) and 0.31 (for 10 μg/mL of anti-HCV IgG and anti-CLDN1); 0.36 (for 1 μg/mL of anti-HCV IgG and anti-SR-BI) and 0.11 (for 10 μg/mL of anti-HCV IgG and anti-SR-BI); 0.6 (for 1 μg/mL of anti-HCV IgG and anti-CD81) and 0.21 (for 10 μg/mL of anti-HCV IgG and anti-CD81), respectively.

TABLE 3
IC₅₀ and CI for each of the combination studied on
HCVcc infection.

| anti-HCV-envelope | anti-HCV- | IC₅₀ | |
|---|---|---|---|
| antibody | receptor antibody | (μg/mL) | CI |
| isotype control | anti-CLDN1 | 0.4 | |
| anti-HCV IgG | anti-CLDN1 | 0.25 | 0.63 |
| isotype control | anti-SRBI | 2.5 | |
| anti-HCV IgG | anti-SRBI | 0.9 | 0.36 |
| isotype control | anti-CD81 | 0.25 | |
| anti-HCV IgG | anti-CD81 | 0.15 | 0.60 |

### Example 3

### Further Characterization in In Vitro Models for HCV Infection

Following screening and identification of mAbs using different HCV genotypes and model systems (HCVcc, HCVpp, hepatoma cell line, primary human hepatocytes), combinations according to the invention will be further characterized by comparative analysis of neutralization in state-of-the-art in vitro models including primary human hepatocytes (Krieger et al., Hepatology, 2010, 51: 1144-1157; Fofana et al., 2010, 139: 953-964). In order to further investigate the mechanism of neutralization, kinetic assays will be used to identify the entry steps targeted by the antibody combinations. For this purpose, HCVpp- and HCVcc-based kinetic assays that allow the distinction between binding and post-binding events as well as cell lines overexpressing entry factors will be used (Haberstroh et al., Gastroenterology, 2008, 135: 1719-1728; Krieger et al., Hepatology, 2010, 51: 1144-1157).

### Example 4

### Characterization in an In Vivo Model for HCV Infection

As a first step to evaluate the antibody combinations according to the present invention, and establish the essential parameters for protection and treatment of HCV infection, the human liver-chimeric SCID/Alb-uPA mouse model will be used in a preclinical study. This model is a well characterized preclinical model for the in vivo assessment of antivirals. Pharmacokinetic and toxicity of selected mAbs combinations in uPA/SCID mice will be examined as previously described (Law et al., Nat. Med., 2008, 14: 25-27; Vanwolleghem et al., Hepatology, 2008, 47: 1846-1855). Briefly, transplanted SCID/Alb-uPA mice will be infected with HCV-infected human serum intravenously and the effect of mAbs combinations on viral load will be assessed. Treatment outcome will be evaluated clinically (toxicity), virologically (viral load), and morphologically (histopathology of transplanted hepatocytes and other tissues) as described recently (Vanwolleghem et al., Gastroenterology, 2007, 133: 1144-1155). The safety profile will be further assessed in non human primates.

### Example 5

### Phase I/IIa Clinical Trials

Following completion of the studies in the uPA-SCID mouse model as well as toxicity studies in non human primates, clinical phase I/IIa trials will be initiated in HCV infected humans resistant or not eligible to standard of care using a longstanding collaboration of Inserm U748-UDS with the Strasbourg Center for Clinical Investigation (CIC) at Strasbourg. Two study designs are required to assess safety and efficacy for prevention and treatment of HCV infection:

Prevention of HCV Infection in Subjects Undergoing Liver Transplantation.

The combination of Abs will be evaluated to prevent the universal re-infection of the liver graft following liver transplantation by achieving a reduction in viral load (as measured quantitatively by HCV RT-PCR) post-transplant by ≧2 log 10 from the baseline value

Treatment of HCV Infection in Subjects Chronically Infected Patients.

The combination of Abs will be evaluated to achieve reduction in viral load by >2 log 10 from the baseline value.

### OTHER EMBODIMENTS

Other embodiments of the invention will be apparent to those skilled in the art from a consideration of the specification or practice of the invention disclosed herein. It is intended that the specification and examples be considered as exemplary only, with the true scope of the invention being indicated by the following claims.

1. A combination of at least one anti-HCV-envelope antibody and at least one anti-HCV-receptor antibody, wherein the anti-HCV-envelope antibody and the anti-HCV-receptor antibody act in synergy to inhibit HCV entry in susceptible cells.

2. The combination according to claim 1, wherein the anti-HCV-envelope antibody and anti-HCV-receptor antibody are monoclonal antibodies or biologically active fragments thereof.

3. The combination according to claim 1, wherein the anti-HCV-envelope antibody is an anti-HCV IgG from an individual chronically or previously infected with HCV.

4. The combination according to claim 1, wherein the anti-HCV-envelope antibody is an anti-HCV-envelope glycoprotein antibody selected from the group consisting of anti-E1 antibodies, anti-E2 antibodies and biologically active fragments thereof.

5. The combination according to claim 1, wherein the anti-HCV-receptor antibody is an antibody against a HCV receptor selected from the group consisting of heparan sulfate, LDL receptor, CD81, SB-RI, Occludin and Claudin-1 or against a region of such a HCV receptor that is involved in HCV entry into susceptible cells.

6. The combination of claim 5, wherein the anti-HCV-receptor antibody is an anti-Claudin 1 antibody that binds to the extracellular domain of Claudin 1.

7. The combination of claim 2, wherein the monoclonal antibodies are humanized, de-immunized or chimeric.

8. The combination of claim 1, wherein at least one of the anti-HCV-envelope antibody and anti-HCV-receptor antibody is attached to a therapeutic agent.

9. The combination of claim 1, wherein the combination index (CI) of the combination is lower than 1.

10. A method for treating or preventing a HCV infection or HCV-related disease in a subject comprising a step of administering an effective amount of the combination of claim 1 to the subject.

11. The method according to claim 10, wherein treating or preventing a HCV infection comprises controlling chronic HCV infection in the subject.

12. The method according to claim 10, wherein treating or preventing a HCV infection comprises preventing HCV re-infection and recurrence in a liver transplantation patient.

13. A pharmaceutical composition comprising a combination according to claim 1 and at least one pharmaceutically acceptable carrier or excipient.

14. The pharmaceutical composition according to claim 13 further comprising at least one anti-viral agent.

15. The pharmaceutical composition according to claim 14, wherein the anti-viral agent is selected from the group consisting of interferons, rabivirin, anti-hepatitis C virus monoclonal antibodies, anti-hepatitis C virus polyclonal antibodies, RNA polymerase inhibitors, protease inhibitors, IRES inhibitors, helicase inhibitors, antisense compounds, ribozymes, and any combination thereof.

16. The combination of claim 6, wherein the anti-HCV-receptor antibody is a monoclonal antibody selected from the group consisting of OM-4A4-D4, OM-7C8-A8, OM-6D9-A6, OM-7D4-C1, OM-6E1-B5, OM-3E5-B6, OM-8A9-A3, OM-7D3-B3 and any biologically active fragment thereof that binds Claudin 1 extracellular domain.

17. The combination of claim 9, wherein the combination index of the combination is lower than 0.75.

18. The combination of claim 17, wherein the combination index of the combination is lower than 0.50.

19. The combination of claim 18, wherein the combination index of the combination is lower than 0.30.
